SEQUENCE LISTING (12 sequences)

SEQ ID NO: 1 - DNA, 175 bp, Homo sapiens
aaagttatgccattttgttttctttctcctcagctctaaatactctgaagtccaaagaagttgtatgttgggtgggctcccttcaactttaacatggaagtgctttctgtgactttaaaagtaagtgcttccatgttttagtaggagtgaatccaatttacttctccaaaataga

SEQ ID NO: 2 - DNA, 277 bp, Homo sapiens
tgccttttacccttctggaggagaacacgaatctttgggaactagttcaggaaggtaaaaaaatttttttcttctaaagttatgccattttgttttctttctcctcagctctaaatactctgaagtccaaagaagttgtatgttgggtgggctcccttcaactttaacatggaagtgctttctgtgactttaaaagtaagtgcttccatgttttagtaggagtgaatccaatttacttctccaaaatagaacacgctaacctcatttgaagggatcc

SEQ ID NO: 3 - DNA, 151 bp, Homo sapiens
acacgctaacctcatttgaagggatcccctttgctttaacatgggggtacctgctgtgtgaaacaaaagtaagtgcttccatgtttcagtggaggtgtctccaagccagcacaccttttgttacaaaatttttttgttattgtgttttaag

SEQ ID NO: 4 - DNA, 233 bp, Homo sapiens
gaatccaatttacttctccaaaatagaacacgctaacctcatttgaagggatcccctttgctttaacatgggggtacctgctgtgtgaaacaaaagtaagtgcttccatgtttcagtggaggtgtctccaagccagcacaccttttgttacaaaatttttttgttattgtgttttaaggttactaagcttgttacaggttaaaggattctaactttttccaagactgggctcc

SEQ ID NO: 5 - DNA, 180 bp, Homo sapiens
gttactaagcttgttacaggttaaaggattctaactttttccaagactgggctccccaccacttaaacgtggatgtacttgctttgaaactaaagaagtaagtgcttccatgttttggtgatggtaagtcttccttttacatttttattatttttttagaaaataactttattgtattga

SEQ ID NO: 6 - DNA, 291 bp, Homo sapiens
tgtctccaagccagcacaccttttgttacaaaatttttttgttattgtgttttaaggttactaagcttgttacaggttaaaggattctaactttttccaagactgggctccccaccacttaaacgtggatgtacttgctttgaaactaaagaagtaagtgcttccatgttttggtgatggtaagtcttccttttacatttttattatttttttagaaaataactttattgtattgaccgcagctcatatatttaagctttattttgtatttttacatctgttaaggggccc

SEQ ID NO: 7 - DNA, 154 bp, Homo sapiens
ccgcagctcatatatttaagctttattttgtatttttacatctgttaaggggccccctctactttaacatggaggcacttgctgtgacatgacaaaaataagtgcttccatgtttgagtgtggtggttcctacctaatcagcaattgcgttaac

SEQ ID NO: 8 - DNA, 241 bp, Homo sapiens
taagtcttccttttacatttttattatttttttagaaaataactttattgtattgaccgcagctcatatatttaagctttattttgtatttttacatctgttaaggggccccctctactttaacatggaggcacttgctgtgacatgacaaaaataagtgcttccatgtttgagtgtggtggttcctacctaatcagcaattgcgttaacgcccacactgtgtgcagttcttggctacagg

SEQ ID NO: 9 - DNA, 660 bp, Homo sapiens
aaagttatgccattttgttttctttctcctcagctctaaatactctgaagtccaaagaagttgtatgttgggtgggctcccttcaactttaacatggaagtgctttctgtgactttaaaagtaagtgcttccatgttttagtaggagtgaatccaatttacttctccaaaatagaacacgctaacctcatttgaagggatcccctttgctttaacatgggggtacctgctgtgtgaaacaaaagtaagtgcttccatgtttcagtggaggtgtctccaagccagcacaccttttgttacaaaatttttttgttattgtgttttaaggttactaagcttgttacaggttaaaggattctaactttttccaagactgggctccccaccacttaaacgtggatgtacttgctttgaaactaaagaagtaagtgcttccatgttttggtgatggtaagtcttccttttacatttttattatttttttagaaaataactttattgtattgaccgcagctcatatatttaagctttattttgtatttttacatctgttaaggggccccctctactttaacatggaggcacttgctgtgacatgacaaaaataagtgcttccatgtttgagtgtggtggttcctacctaatcagcaattgcgttaac

SEQ ID NO: 10 - DNA, 766 bp, Homo sapiens
tgccttttacccttctggaggagaacacgaatctttgggaactagttcaggaaggtaaaaaaatttttttcttctaaagttatgccattttgttttctttctcctcagctctaaatactctgaagtccaaagaagttgtatgttgggtgggctcccttcaactttaacatggaagtgctttctgtgactttaaaagtaagtgcttccatgttttagtaggagtgaatccaatttacttctccaaaatagaacacgctaacctcatttgaagggatcccctttgctttaacatgggggtacctgctgtgtgaaacaaaagtaagtgcttccatgtttcagtggaggtgtctccaagccagcacaccttttgttacaaaatttttttgttattgtgttttaaggttactaagcttgttacaggttaaaggattctaactttttccaagactgggctccccaccacttaaacgtggatgtacttgctttgaaactaaagaagtaagtgcttccatgttttggtgatggtaagtcttccttttacatttttattatttttttagaaaataactttattgtattgaccgcagctcatatatttaagctttattttgtatttttacatctgttaaggggccccctctactttaacatggaggcacttgctgtgacatgacaaaaataagtgcttccatgtttgagtgtggtggttcctacctaatcagcaattgcgttaacgcccacactgtgtgcagttcttggctacagg

SEQ ID NO: 11 - DNA, 889 bp, Homo sapiens
aaagttatgccattttgttttctttctcctcagctctaaatactctgaagtccaaagaagttgtatgttgggtgggctcccttcaactttaacatggaagtgctttctgtgactttaaaagtaagtgcttccatgttttagtaggagtgaatccaatttacttctccaaaatagaacacgctaacctcatttgaagggatcccctttgctttaacatgggggtacctgctgtgtgaaacaaaagtaagtgcttccatgtttcagtggaggtgtctccaagccagcacaccttttgttacaaaatttttttgttattgtgttttaaggttactaagcttgttacaggttaaaggattctaactttttccaagactgggctccccaccacttaaacgtggatgtacttgctttgaaactaaagaagtaagtgcttccatgttttggtgatggtaagtcttccttttacatttttattatttttttagaaaataactttattgtattgaccgcagctcatatatttaagctttattttgtatttttacatctgttaaggggccccctctactttaacatggaggcacttgctgtgacatgacaaaaataagtgcttccatgtttgagtgtggtggttcctacctaatcagcaattgcgttaacgcccacactgtgtgcagttcttggctacaggccattactgttgctaatatgcaactctgttgaatataaattggaattgcactttagcaatggtgatggagctatcttttagatctccagtatccttttcctcttcagtagagatttctatatcttgaaataaataaaaaagctctccgttaagacatggggctgtgaaatgggtgaaacagaagccaattaaaaccta

SEQ ID NO: 12 - DNA, 1094 bp, Homo sapiens
tgccttttacccttctggaggagaacacgaatctttgggaactagttcaggaaggtaaaaaaatttttttcttctaaagttatgccattttgttttctttctcctcagctctaaatactctgaagtccaaagaagttgtatgttgggtgggctcccttcaactttaacatggaagtgctttctgtgactttaaaagtaagtgcttccatgttttagtaggagtgaatccaatttacttctccaaaatagaacacgctaacctcatttgaagggatcccctttgctttaacatgggggtacctgctgtgtgaaacaaaagtaagtgcttccatgtttcagtggaggtgtctccaagccagcacaccttttgttacaaaatttttttgttattgtgttttaaggttactaagcttgttacaggttaaaggattctaactttttccaagactgggctccccaccacttaaacgtggatgtacttgctttgaaactaaagaagtaagtgcttccatgttttggtgatggtaagtcttccttttacatttttattatttttttagaaaataactttattgtattgaccgcagctcatatatttaagctttattttgtatttttacatctgttaaggggccccctctactttaacatggaggcacttgctgtgacatgacaaaaataagtgcttccatgtttgagtgtggtggttcctacctaatcagcaattgcgttaacgcccacactgtgtgcagttcttggctacaggccattactgttgctaatatgcaactctgttgaatataaattggaattgcactttagcaatggtgatggattgttaagccaatgacagaatttaaaccacagacttactttgatagcactcttaatggtataacttcttctcccattttatgtctctctttatgttttttcttatgtttcctttttgttttcaagagagagctatcttttagatctccagtatccttttcctcttcagtagagatttctatatcttgaaataaataaaaaagctctccgttaagacatggggctgtgaaatgggtgaaacagaagccaattaaaaccta

(19) United States
(12) Patent Application Publication
(10) Pub. No.: US 2018/0371423 A1
(43) Pub. Date: Dec. 27, 2018

(54) NON-VIRAL IPSCS INDUCING METHOD, COMPOSITIONS, KITS AND IPSCS

(71) Applicants: Linli Wang, Guangzhou (CN); Guangzhou Biocare Biotechnology Co., Ltd, Guangzhou (CN)

(72) Inventors: Linli Wang, Guangzhou (CN); Yuehua Chen, Guangzhou (CN); Libing Song, Guangzhou (CN); Jian Mo, Guangzhou (CN)

(73) Assignees: Guangzhou Biocare Biotechnology Co., Ltd, Guangzhou (CN); Linli Wang, Guangzhou (CN)

(21) Appl. No.: 16/106,039

(22) Filed: Aug. 21, 2018

Related U.S. Application Data
(62) Division of application No. 15/393,288, filed on Dec. 29, 2016.

(51) Int. Cl. C12N 5/074; C12N 15/85

(52) U.S. Cl. CPC C12N 5/0696; C12N 2820/60; C12N 2800/108; C12N 2501/727; C12N 2501/604; C12N 2501/602; C12N 2501/065; C12N 15/85

(57) ABSTRACT

The present invention relates to a non-viral iPSCs induction method as well as the compositions, kits and iPSCs obtained therefrom. More specifically, the induction method comprises the following steps: 1) Constructing a recombinant plasmid by introducing the DNA sequences expressing the reprogramming factors POU5F1, SOX2, GLIS1, KLF4, MYCL and hsa-miR-302s into an episomal vector; 2) Obtaining iPSCs by introducing the recombinant plasmids obtained in step 1) into human somatic cells, and reprogramming induction culture of the cells. The method reduces the risk of clinical applications of iPSCs by using a combination of highly-safe reprogramming factors without the introduction of high-risk reprogramming factors such as c-MYC, SV40-LT and TP53 inhibitors; The method is highly applicable.

### CROSS REFERENCE TO RELATED APPLICATIONS

This application is a divisional application of application Ser. No. 15/393,288 filed on Dec. 29, 2016, which is based upon and claims priority to Chinese Patent Application No. 201610411378.X, filed on Jun. 13, 2016, the entire contents of which are incorporated herein by reference.

### REFERENCE TO SEQUENCE LISTING

The Sequence Listing is submitted concurrently with the specification as an ASCII formatted text file via EFS-Web, with a file name of “Sequence_Listing_JILY-1658-USPT.TXT”, a creation date of Dec. 28, 2016, and a size of 7988 bytes. The Sequence Listing filed via EFS-Web is part of the specification and is incorporated in its entirety by reference herein.

### TECHNICAL FIELD

The present invention relates to a non-viral iPSCs induction method as well as the compositions, kits and iPSCs obtained therefrom. The invention relates to the field of bio-engineering technology and regenerative medicine.

### BACKGROUND

Cell differentiation was considered to be unidirectional and irreversible in the classical stem cell biology, traditionally. Until 1962, John B. Gurdon, an English scientist, successfully introduced an intact nuclear from an intestinal cell into an enucleated egg of a Xenopus and obtained a living tadpole. It was the first evidence that the nuclear of a somatic cell could be reprogrammed to a pluripotent cell in the early development stage of an embryo. Various cloned mammals were created by similar technologies since then. 40 years after Gurdon's report, Shinya Yamanaka, a Japanese scientist, successfully reprogrammed the mouse fibroblasts into the induced pluripotent stem cells (iPSCs) with the pluripotency of the embryonic stem cells (ESCs) by using four specific genes encoding transcription factors (OCT4 (POU5F1 as gene name), SOX2, KLF4 and c-Myc, which referred to as OSKM) carried by the retroviruses in 2006, and Yamanaka's research team obtained human iPSCs by the same method in the next year. At the same time, the James A. Thomson's team also successfully obtained human iPSCs (hiPSCs) via four additional transcription factors (OCT4, SOX2, Nanog, Lin28, referred to as OSNL) carried by the lentivirus. The advent of iPSCs brought an unprecedented revolution to the stem cells and the regenerative medicine, since iPSCs are not limited by the ethical restrictions of using cloning technique and ESCs (derived from embryonic tissues), and they are highly similar to, or even indistinguishable from ESCs in terms of morphology, gene expression profiling, epigenetic lineage, self-renewal capacity and pluripotency. In addition, iPSCs can be differentiated to all types of adult cells, tissues and organs, thus they can play an important role in the fields of transplantation of organs, tissues and cells, as well as cancer treatment, repair of inherited disease, and drug screening.

The introduction of reprogramming factors into cells can be divided into a viral and nonviral-mediated method. The introduction of reprogramming genes by using virus is a classical method, however, there might be chromosomal instability or even cell carcinogenesis possibilities, due to the insertion and integration of the lentivirus or retrovirus genome into the host genome. Stadtfeld et al. obtained the mouse iPSCs (miPSCs) by a non-integrated adenoviral vector carrying four factors in 2008. Fusaki et al. obtained a success derivation of hiPSCs by using Sendai virus in the absence of integration into the genome in 2009. Nevertheless, active viruses are still limited to the experimental studies for their unknown clinical risks. Okita et al. reported a successful derivation of miPSCs with an ordinary eukaryotic expressed plasmid in 2008. However, ordinary plasmids are easily to be lost, which requires a multiple transfection, resulting in a very low induction efficiency. Thus, it is difficult to obtain hiPSCs, and it could not be widely used in the related studies. Junying Yu reported a derivation of hiPSCs with the use of a non-integrated episomal vector carrying reprogramming factors in 2009. The episomal vector contains two DNA elements, OriP/EBNA1 (Epstein-Barr nuclear antigen-1) (The episomal vectors in the instant disclosure are all episomal vectors containing OriP/EBNA1 elements), wherein the expression product of the EBNA1 gene could bind with OriP element, and it makes the episomal vectors more efficiently replicable than the ordinary plasmids within cells. Therefore, the episomal vectors only need to be transfected once in the reprogramming process, however, they could be completely lost in about 2 months. So far, the technology has been widely used in non-integrated induced reprogramming of somatic cells. However, it has been reported that all of the widely used somatic cell reprogramming systems which use the episomal vectors contain at least one of the many high-risk oncogenes or factors, such as c-MYC, SV40-LT, TP53 inhibitor and other carcinogenic factors. There may be risks in the iPSCs obtained using these high-risk factors, such as tumorigenesis of cells. It is necessary to acquire technologies with high safety and suitable for a large-scale preparation of the iPSCs, before a wide range of clinical applications of the iPSCs.

### SUMMARY

In order to overcome the shortcomings in the prior art, an objective of the instant disclosure is to provide a non-viral iPSCs induction method suitable for clinical applications with high safety.

The object of the instant disclosure can be attained by adopting the following technical scheme:

A non-viral iPSCs induction method which comprises the following steps:

1) Constructing a recombinant plasmid by introducing the DNA sequences expressing the reprogramming factors POU5F1, SOX2, GLIS1, KLF4, MYCL and hsa-miR-302s into an episomal vector;

wherein, the hsa-miR-302s DNA sequence comprises one or more sequences selected from hsa-miR-302a, hsa-miR-302b, hsa-miR-302c and hsa-miR-302d;

2) Obtaining iPSCs by introducing the recombinant plasmids obtained in step 1) into human somatic cells and reprogramming the induction culture of the cells.

Preferably, the hsa-miR-302s DNA sequence in step 1) further comprises a DNA sequence of hsa-miR-367.

Preferably, the link and transcription initiation of POU5F1, SOX2, GLIS1, KLF4 and MYCL in step 1) are through a RNA polmerase II promoter (pol II promoter).

Preferably, the link and transcription initiation of POU5F1, SOX2, GLIS1, KLF4 and MYCL in step 1) are through a promoter selected from EF-1α promoter, CMV promoter and CAG promoter.

Preferably, the link and transcription initiation of hsa-miR-302s in step 1) are through a promoter selected from RNA polmerase I promoter (pol I promoter), RNA polmerase II promoter (pol II promoter) and RNA polmerase III promoter (pol III promoter).

Preferably, the link and transcription initiation of hsa-miR-302s in step 1) are through a promoter selected from CMV promoter, U6 promoter and H1 promoter.

Preferably, in step 1), the reprogramming factors POU5F1, SOX2, GLIS1, KLF4 and MYCL are selected from IRES and 2A-based coexpression elements, and the genes of the reprogramming factors expressing two or more proteins are coexpressed through a single promoter.

Preferably, in step 1), the reprogramming factors POU5F1, SOX2, GLIS1, KLF4 and MYCL are selected from IRES1, IRES2, P2A and F2A coexpression elements, and the genes of the reprogramming factors expressing two or more proteins are coexpressed through a single promoter.

Preferably, in step 1), the reprogramming factors POU5F1 and GLIS1 are linked through a P2A coexpression element and the transcription initiation is through an EF-1a promoter, the reprogramming factors KLF4 and SOX2 are linked through a P2A coexpression element and the transcription initiation is through an EF-1α promoter, the DNA sequences containing genes of SOX2, GLIS1, KLF4 and POU5F1 are constructed into an episomal vector together; the transcription initiation of reprogramming factor MYCL and the transcription initiation of reprogramming factor hsa-miR-302s are through an EF-1a promoter and a CMV promoter respectively, and then the DNA sequences are constructed into an episomal vector.

Preferably, a small molecule compound is added during the induction culture in step 2), the small molecule compound is one or more molecules selected from MEK inhibitors, GSK-3β inhibitors, histone deacetylase inhibitors and lysine specific demethylasel inhibitors.

Preferably, the small molecule compound in step 2) is a combination of PD0325901, CHIR-99021, sodium butyrate and tranylcypromine hydrochloride.

Preferably, in step 2), the concentration of PD0325901 is in a range of 0.1-2 μM, the concentration of CHIR-99021 is in a range of 0.1-6 μM, the concentration of sodium butyrate is in a range of 0.05-2 μM, and the concentration of tranylcypromine hydrochloride is in a range of 0.1-10 μM.

Preferably, in step 2), the above-mentioned small molecule compound is added on any one or more days from day 0 to day 12 during the induction culture.

Preferably, in step 2), the above-mentioned small molecule compound is added on every day from day 0 to day 8 during the induction culture.

It is another object of the instant disclosure to provide an induction composition including recombinant plasmids for using in the above method, the recombinant plasmids are obtained by constructing the DNA sequences expressing the reprogramming factors POU5F1, SOX2, GLIS1, KLF4, MYCL and hsa-miR-302s into an episomal vector.

Preferably, the induction composition further comprises a small molecule compound, the small molecule compound is one or more molecules selected from MEK inhibitors, GSK-3β inhibitors, histone deacetylase inhibitors and lysine specific demethylasel inhibitors.

It is an object of the instant disclosure to provide a kit comprising the above-described induction composition.

It is an object of the instant disclosure to provide the iPSCs obtained by the above-described method.

Compared with the prior art, the invention has the advantages that:

1) The present invention provides a non-viral induction method of iPSC which reduces the risk of clinical applications of iPSCs by using a combination of highly safe reprogramming factors without an introduction of high-risk reprogramming factors such as c-MYC, SV40-LT and TP53 inhibitors;

2) The present invention provides a non-viral induction method of iPSC with high applicability, it can successfully induce iPSCs without the limitation from the types of promoters, the number of episomal vectors, the coexpression elements or the length of hsa-miR-302s precursors;

3) The small molecule compounds provided by the present invention can effectively shorten the induction culture time, and they can be stimulated within at least two days during the whole induction process, thus the iPSCs could be successfully and efficiently obtained therefrom.

### BRIEF DESCRIPTION OF THE DRAWINGS

FIG. 1 is a schematic diagram of the pCEP4 plasmid;

FIG. 2 is a schematic diagram of the recombinant plasmid in Example 1;

FIG. 3 is a microscopic view of Example 1;

FIG. 4 shows the karyotype identification of chromatins in Example 1;

FIG. 5 shows the teratoma identification in Example 1;

FIG. 6 shows an identification of pluripotent molecular markers in Example 1;

FIG. 7 shows the AP staining results of the experimental group and the control group in Example 2;

FIG. 8 is a column diagram which shows the detecting rate of karyotypic abnormalities in Example 3;

FIG. 9 is a scanning image of AP staining in Example 4;

FIG. 10 is a column diagram which shows the counting of AP staining in Example 4;

FIG. 11 is a scanning image of AP staining in Example 5.

FIG. 12 is a column diagram which shows the counting of AP staining in Example 5;

FIG. 13 is a scanning image of AP staining in Example 6;

FIG. 14 is a column diagram which shows the counting of AP staining in Example 6;

FIG. 15 is a scanning image of AP staining in Example 7;

FIG. 16 is a column diagram which shows the counting of AP staining in Example 7;

FIG. 17 is a scanning image of AP staining in Example 8;

FIG. 18 is a column diagram which shows the counting of AP staining in Example 8;

FIG. 19 is a scanning image of AP staining in Example 9;

FIG. 20 is a column diagram which shows the counting of AP staining in Example 9;

FIG. 21 is a schematic diagram of the recombinant plasmid from Group 1 in Example 10;

FIG. 22 is a schematic diagram of the recombinant plasmid from Group 3 in Example 10;

FIG. 23 is a schematic diagram of the recombinant plasmid from Group 4 in Example 10;

FIG. 24 is a schematic diagram of the recombinant plasmid from Group 5 in Example 10;

FIG. 25 is a scanning image of AP staining in Example 10;

FIG. 26 is a column diagram which shows the counting of AP staining in Example 10.

### DETAILED DESCRIPTION OF THE EMBODIMENTS

The instant disclosure will be described in further detail in consideration of the following description of various embodiments and the accompanying drawings.

We have developed a technique for obtaining non-integrated iPSCs by using episomal vectors and a combination of highly safe reprogramming factors, as well as obtaining hiPSCs efficiently under the stimulation of a very short and low-risk small molecule compound, without the introduction of high-risk reprogramming factors such as c-MYC, SV40-LT and TP53 inhibitors, making the acquisition of non-integrated iPSCs better meet the clinical safety level and the scale of production needs.

In the following embodiments, all the reagents, plasmids and genes used are commercially available or could be obtained through conventional experimental methods, unless otherwise specified.

Information of the reagents in the following embodiments, including: PD0325901 (CAS No. 391210-10-9), PD98059 (CAS No. 167869-21-8), Tideglusib (CAS No. 865854-05-3), 1-Azakenpaullone (CAS No. 676596-65-9), CHIR-99021 (CAS No. 252917-06-9), TDZD-8 (CAS No. 327036-89-5), TWS119 (CAS No. 601514-19-6), AR-A014418 (CAS No. 487021-52-3), AZD2858 (CAS No. 486424-20-8), IM-12 (CAS No. 1129669-05-1), M 344 (CAS No. 251456-60-7), NCH 51 (CAS No. 848354-66-5), NSC 3852 (CAS No. 3565-26-2; 5-Nitroso-8-hydroxyquinoline), Sodium Phenylbutyrate (CAS No. 1716-12-7), Pyroxamide (CAS No. 382180-17-8; N-Hydroxy-N-3-pyridinyloctanediamide), SBHA (CAS No. 38937-66-5, Suberohydroxamic acid), Scriptaid (CAS No. 287383-59-9), Sodium Butyrate (CAS No. 156-54-7), Sodium valproate (CAS No. 1069-66-5), Pifithrin-μ (CAS No. 64984-31-2), Pifithrin-α hydrobromide (CAS No. 63208-82-2), Tranylcypromine hydrochloride (CAS No. 1986-47-6).

The methods provided here in this application are suitable for most of the human somatic cells or adult stem cells (also known as adult cells), including but not limited to the renal epithelial cells. In the following embodiments, the cells used in the examples were all derived from urine samples, except for those marked as suitable for a variety of cell reprogramming; The human somatic cells used were urine-derived renal epithelial cells, which obtained through the centrifugation of human urine and the amplification and culture of the collected renal epithelial cells. All urine donors have signed an informed consent approved by the ethics committee from Guangzhou Biocare Cancer Institute.

The methods provided herein by the present invention are also applicable to the adult cells such as human fibroblasts or human mesenchymal stem cells, which are all commercially available.

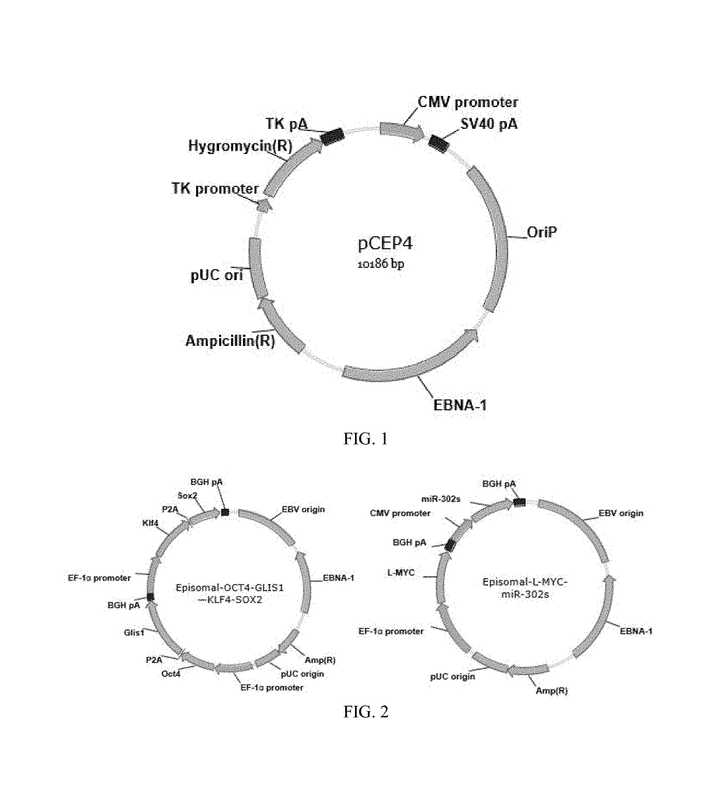

In the present application, the term “episome” in the episomal vector (or plasmid) is an episomal type (plasmid or vector), a free plasmid (plasmid or vector), which is derivative from the adjective “Episomal”. In the following embodiments, the episomal vectors used were Episomal-EBNA1/OriP plasmids which derived from Invitrogen pCEP4 Mammalian Expression Vector with a Product No. V04450, wherein the structure is shown in FIG. 1. In the present application, POU5F1 is also known as OCT4; MYC is also known as L-MYC.

In the following embodiments, the gene name, organism, accession number and length of the reprogramming factors are shown as follows,

TABLE 1
Gene, organism, accession number and length of the reprogramming
factors

| | | | Accession | Length |
|---|---|---|---|---|
| Gene | Aliases | Organism | number | (bp) |
| POU5F1 | Oct4, | Homo sapiens | NM_002701 | 1083 |
| | OCT3, OCT4, | | | |
| | OTF3, OTF4, | | | |
| | OTF-3, Oct-3, | | | |
| | Oct-4 | | | |
| GLIS1 | Null | Homo sapiens | NM_147193 | 1863 |
| KLF4 | EZF, GKLF | Homo sapiens | NM_004235 | 1440 |
| | | | NM_004235 | 1413 |
| SOX2 | ANOP3, | Homo sapiens | NM_003106 | 954 |
| | MCOPS3 | | | |
| MYCL | LMYC, | Homo sapiens | NM_001033081 | 1095 |
| | L-MYC, | | | |
| | MYCL1, | | | |
| | bHLHe38 | | | |
| hsa-miR- | Null | Homo sapiens | See below | See |
| 302s | | | | below |

In the following embodiments, the information of hsa-miR-302s is as follows:

TABLE 2
information of hsa-miR-302s

| | | Accession number | Accession number |
|---|---|---|---|
| | Accession | of | of |
| Name | number | 5P Mature Sequence | 3P Mature Sequence |
| hsa-miR-302a | MI0000738 | MIMAT0000683 | MIMAT0000684 |
| hsa-miR-302b | MI0000772 | MIMAT0000714 | MIMAT0000715 |
| hsa-miR-302c | MI0000773 | MIMAT0000716 | MIMAT0000717 |
| hsa-miR-302d | MI0000774 | MIMAT0004685 | MIMAT0000718 |
| hsa-miR-367 | MI0000775 | MIMAT0004686 | MIMAT0000719 |

In the following embodiments, the recombinant plasmids were constructed with hsa-miR-302s of different lengths, wherein the information of hsa-miR-302s of different lengths is as follows: Sequence hsa-miR-302b (5′ +75 bp, +3′ +27 bp) is shown as SEQ ID No:1;

Sequence hsa-miR-302b (5′ +150 bp, 3′ +54 bp) is shown as SEQ ID No:2;

Sequence hsa-miR-302c (5′ +27 bp, 3′ +56 bp) is shown as SEQ ID No:3;

Sequence hsa-miR-302c (5′ +54 bp, 3′ +111 bp) is shown as SEQ ID No:4;

Sequence hsa-miR-302a (5′ +55 bp, 3′ +56 bp) is shown as SEQ ID No:5;

Sequence hsa-miR-302a (5′ +111 bp, 3′ +111 bp) is shown as SEQ ID No:6;

Sequence hsa-miR-302d (5′ +55 bp, 3′ +31 bp) is shown as SEQ ID No:7;

Sequence hsa-miR-302d (5′ +111 bp, 3′ +62 bp) is shown as SEQ ID No:8;

Sequence hsa-miR-302bcad (5′ +75 bp, 3′ +31 bp) is shown as SEQ ID No:9;

Sequence hsa-miR-302bcad (5′ +150 bp, 3′ +62 bp) is shown as SEQ ID No:10;

Sequence hsa-miR-302cluster (5′ +75 bp, 3′ +130 bp) is shown as SEQ ID No:11; Sequence hsa-miR-302cluster (5′ +150 bp, 3′ +260 bp) is shown as SEQ ID No:12;

In the following embodiments, the comparison experiment were performed by constructing c-MYC, SV40-LT and TP53 shRNA, a TP53 inhibitor, into the episomal vectors or by TP53 siRNA transfection for TP53 inhibitor. Wherein, the two TP53 inhibitors, TP53 shRNA1 and TP53 shRNA2, were constructed into the episomal vectors respectively and tested by electroporation. TP53 siRNA1 and TP53 siRNA2 were transfected by liposome transfection;

Wherein,

The target of TP53 shRNA1 is 5′-GACTCCAGTGGTAATCTAC-3′;

The target of TP53 shRNA2 is 5′-GTCCAGATGAAGCTCCCAGAA-3′;

TP53 siRNA1 was purchased from Santa Cruz Biotechnology, Product No. SC-45917;

TP53 siRNA2 was purchased from Cell Signalling Technology, Product No. #6231.

In the following embodiments, whether the iPSCs could be formed and whether their chromosome stability, self-renewal and pluripotency could be maintained were assessed by AP staining, analysis of karyotype and teratoma, as well as the pluripotency assessment by flow cytometry (FACS).

1. The procedure for AP staining is as follows,

a) Aspirate and remove the growth medium from the cultures to be stained when the cell culture is finished, wash the culture with 1×PBS one time; Fix the cells with 4% paraformaldehyde at room temperature for 2 min;

b) Aspirate the fixing solution, wash the culture with 1×TBST for 3 times; Balance with AP buffer at room temperature for 5 min;

c) Chromogenic reaction was performed with AP chromogenic reagent at room temperature in dark for 15 min (5-15 min, terminated when the color of clones became darker and without background, increase the reaction time appropriately if the clones were not stained). The chromogenic reagent was aspirated, cells were washed with 1×PBS for two times, covered with an appropriate amount of 1×PBS, observed and counted under a microscope.

Alkaline phosphatase (AP) is a phosphomonoesterase. The alkaline phosphatase in the cytoplasm can hydrolyze sodium naphthol phosphate to produce α-naphthol in alkaline environments. The latter reacts with a stable azo salt and presents a deep purple color, which the presence of alkaline phosphatase and the abundance of expression could be determined accordingly. Alkaline phosphatase is highly expressed in undifferentiated pluripotent stem cells, and the activity of alkaline phosphatase in the differentiated pluripotent stem cells is decreased. Therefore, whether the cells are clones of iPSCs could be determined by alkaline phosphatase staining (AP staining). And, thus the efficiency of iPSCs production can be easily judged according to the efficiency of AP positive clones.

In Example 1-2 and 4-6, the efficiency of AP positive clones was determined based on the number of cells in each group after electroporation transformation, ie the efficiency of AP positive clones=number of AP positive clones/number of passaged cells per well after electroporation in each group;

In Example 3 and 7-10, the efficiency of AP positive clones was determined based on the total number of cells after electroporation transformation, i.e. the efficiency of AP positive clones=number of AP positive clones/total number of cells after electroporation transformation.

Karyotype Identification

1) Experimental Reagents

20 μg/mL colchicine; PBS; saline; 0.25% trypsin; 0.075M potassium chloride solution; MEF; Carnoy's fixative; Giemsa staining solution; 3% Tris.

2) Experimental Appliances

37° C. incubators; micro pipette (100 μl, 1 mL); conventional centrifuge; thermostatic water bath; slides; plastic Turkey Baster; oven; pickling bath, staining bath; microscope.

3) Experimental Procedure

3.1. Cell Preparation

Cells were grown in a good state without differentiation, and reached a 80%-90% confluence.

3.2) Treatment with Colchicine

20 μg/mL colchicine solution was added in the culture medium to reach a final concentration of 0.2 μg/mL before the termination of the cell culture, cells were treated with colchicine in a 37° C. incubator for 100-130 min.

3.3) Hypotonic Treatment

After the treatment with colchicine, the culture medium was aspirated, cells were washed twice with PBS, 0.5 mL 0.25% trypsin was added for digestion, the attached cells were detached by tapping the Petri dish gently, 1 mL MEF was added to stop the digestion, cells were transferred to a 15 mL centrifuge tube by a pipette, centrifuged (1200 rpm, 5 min) and collected. Then, 7 mL KCL solution pre-heated at 37° C. in a concentration of 0.075 mol/L was added, and cells were mixed in a suspension with a pipet, placed in a 37° C. water bath for 18-28 min.

3.4) Pre-Fixation

1 mL fresh prepared Carnoy's fixative (the ratio of methanol to acetic acid is 3:1 in preparation) was added with a plastic Turkey Baster for a pre-fixation for 3 min.

3.5) Fixation

After pre-fixation, cells were centrifuged at 1200 r/min for 5 min, the supernatant was discarded, and about 7 mL of fresh fixative solution was added and mixed well with a plastic head dropperplastic Turkey Baster gently, and fixed for 40 min at 37° C.

3.6) Dropping

After fixation, cells were centrifuged at 1200 r/min for 5 min, then most of the fixative was aspirated with a plastic Turkey Baster, cells were then resuspended in the residual fixative (volume of the residual fixative was determined according to the number of cells), the cell solution were dropped onto a slide with a distance of 30 cm. Note that the glass slide used should be clean.

3.7) Slide Heating

The slides were heated in a drying oven at 75° C. for 3 h immediately after the dropping step.

3.8) Staining (G-Banding)

0.03 g of trypsin powder was added into 55 mL saline, and then be shaken gently, pH 7.2 was adjusted with 3% Tris-solution. The slides were immersed into a trypsin digesting solution for 8 seconds, and then placed into a clean saline solution quickly to terminate their digestion, and then placed in Giemsa staining solution for 5-10 min, and then clamped out of the solution with tweezers, rinsed gently with water on both sides, dried at room temperature or with a dryer.

3.9) Observation Under a Microscope

The dry slides were examined under a microscope, cells were observed firstly at a low magnification for a good split, then observed at a high magnification with an oil immersion objective.

3.10) Analysis (analysis of chromosome number, band type), 20 divisional fields of view were analyzed for each cell sample. If the number of chromosomal abnormalities occurs 3 or more times, it should be judged as abnormal.

Pluripotency (marker) assessment by FACS

1) Cells were digested with 0.25% trypsin, centrifuged, resuspended in PBS, and transferred to a 1.5 mL EP tube.

2) 200 μl 1% paraformaldehyde was added at 37° C., and cells were fixed for 5-10 min.

3) Cells were centrifuged and washed once with PBS, then 200 μl of 90% pre-cooled methanol was added in the tube, cells were left on ice for 30 min.

4) Cells were centrifuged and washed twice with PBS. 50 μL primary antibody solution (1:50 dilution of the antibody) was added at 37° C., and incubated for 30 min.

5) Cells were centrifuged, washed 1-2 times with PBS, and 100 μL secondary antibody solution (1:500 dilution of the antibody) was added at 37° C. in the dark, and incubated for 30 min.

6) Cells were washed once with PBS, resuspended in 300 μL PBS, filtered, loaded in a flow cytometer, and cells with positive signals in the 488 nm (green) or 568 nm (red) channels were collected.

FACS was used in the invention for an assessment of the expression of pluripotent markers OCT4, SSEA4, Tra-1-60 and Tra-1-81;

Wherein, 1) OCT4 is the most core transcription factor of pluripotent stem cells, and is rarely expressed or in a very low expression in differentiated or other adult stem cells. It is the most important molecular marker for the pluripotent stem cells.

SSEA4 is a stage-specific embryonic antigen expressed on the surface of human pluripotent stem cells and which is a glycolipid epitope. The differentiation of human pluripotent stem cell leads to a decrease in the SSEA4 expression, and thus it is often used as a feature of the molecule marker of pluripotent stem cells.

Tra-1-60 and Tra-1-81 are glycoprotein antigens with high molecular weight, which are surface antigens expressed in the pluripotent stem cells and used as the pluripotent molecular markers of pluripotent stem cells.

Therefore, the expression of OCT4, SSEA4, Tra-1-60, Tra-1-81 antigens can be identified by FACS to characterize the molecular markers of human pluripotent stem cells, including iPSCs.

Teratoma Identification

1) When cells reached a 75%-80% confluence, cells were digested with type IV collagenase for 10 min, washed with DMEM/F12 media for 3 times gently, and scraped off by a mechanical method.

2) DMEM/F12 was added, and then cells were centrifuged at 100 g for 5 min.

3) Matrigel and DMEM/F12 were mixed in 1:2 on ice, and then mixed with the cells.

4) The mixture was injected into the muscle or subcutaneous tissue of the limbs of NOD-SCID mice, then the teratomas were taken when they reached a certain size, and the tumor were stained by HE staining and analyzed.

5) Analysis of Three Germ Layers after Teratoma Staining:

Ectoderm: differentiation of melanocytes; differentiation of radially arranged nerve tissue and so on.

Mesoderm: differentiation of muscle tissue; differentiation of cartilage tissue; differentiation of adipose tissue and so on.

Endoderm: differentiation of adenocarcinoma; differentiation of luminal intestinal epithelium and so on.

### Example 1

Example 1 provides a non-viral iPSCs induction method including the following steps:

1) Constructing a recombinant plasmid by introducing the DNA sequences expressing the reprogramming factors OCT4, SOX2, GLIS1, KLF4, L-MYC and hsa-miR-302s into an episomal vector;

Wherein the hsa-miR-302s is hsa-miR-302cluster, sequence of hsa-miR-302cluster is shown as SEQ ID No:12;

Wherein, the reprogramming factors OCT4 and GLIS1 are linked through P2A coexpression element and the transcription initiation is through EF-1α promoter, the reprogramming factors KLF4 and SOX2 are linked through P2A coexpression element and the transcription initiation is through EF-1α promoter, the DNA sequences containing genes of OCT4, GLIS1, KLF4 and SOX2 are constructed into an episomal vector together; the transcription initiation of reprogramming factor L-MYC and the transcription initiation of reprogramming factor hsa-miR-302s are through EF-1α promoter and CMV promoter respectively, and then the DNA sequences are constructed into an episomal vector; the schematic diagram of the recombinant plasmid is shown in FIG. 2;

2) The recombinant plasmids obtained in step 1) were introduced into human somatic cells, and induced to iPSCs after induction culture for 15 days; The human somatic cells were renal epithelial cells isolated from the urine.

In Step 2), the detail operations were as follows:

a) Electroporation transformation: The recombinant plasmids obtained in step 1) were added into the culture of renal epithelial cells after digestion with trypsin, and then transferred into the renal epithelial cells. After electroporation, the cells were seeded on a cell culture plate coated with extracellular matrix;

Matrigel or other extracellular matrix of pluripotent stem cell culture could be used for coating the cell culture plate; Usually, 2-10 μg recombinant plasmids and 0.5-4 million renal epithelial cells were taken from one culture system;

b) Induction culture: 1-3 days after Step 1), or when the cells reached a confluence of 30% or more, the culture medium of pluripotent stem cell was used to continue induction culture. About 15-30 days after the mature of iPSCs clones, the positive clones of iPSCs were identified by AP staining, and the efficiency of AP positive clones was calculated accordingly;

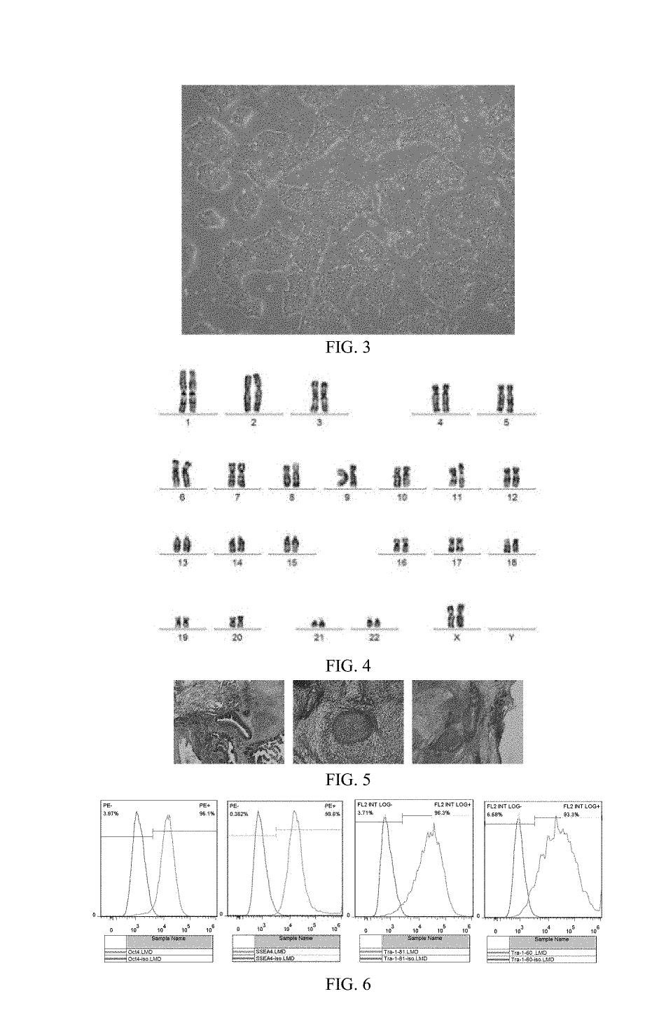

It could be detected from the AP staining that AP positive clones reached 38/2×10⁵ Cells per well. FIG. 3 shows a microscopic field view of iPSCs (4× objective), it shows that the morphology of iPSCs obtained according to the present method in the example was consistent with that of an embryonic stem cell, and they had an ability to self-renewal in vitro.

During the reprogramming of somatic cells, the chromosomal (chromatin) karyotype of the induced iPSCs was abnormal due to various factors, which may lead to the occurrence of tumor or other cell abnormalities. G-banding karyotype analysis was performed to determine whether the karyotype of iPSCs was normal by chromosomal banding (ie, G-banding) after Giemsa staining, and then the analysis of counting, pairing and aligning the chromosomes. The karyotype of the iPSCs obtained in this example is shown in FIG. 4, and the result shows that the karyotype of the iPSCs is normal, indicating that iPSCs with normal karyotype can be obtained by the method provided in this example;

IPSCs, like other pluripotent stem cells, have the ability to differentiate into all types of cells in all three germ layers. Pluripotent stem cells injected subcutaneously or intramuscularly in immunodeficient mice can differentiate into teratomas with three germ layers spontaneously. And thus their pluripotency of as a pluripotent stem cell (such as the differentiation ability to cells of all three germ layers) was determined accordingly. FIG. 5 shows the teratoma identification, indicating that the iPSCs obtained in this example can be differentiated to cells of all three germ layers, which are endoderm (intestinal-like epithelium differentiation), mesoderm (cartilage differentiation) and ectoderm (radially arranged nerve tissue and melanocytes) from left to right in the figure, and thus their pluripotency was indicated;

FIG. 6 shows an identification of pluripotent molecular markers. The expression of OCT4, SSEA4, Tra-1-60 and Tra-1-81 in iPSCs were identified by FACS. It shows that the molecular markers for pluripotency were all above 90%, which indicated that the iPSCs obtained by the method possess molecular marker characteristics of pluripotent stem cells.

### Example 2

This example is based on Example 1, where in the induction process of step 2), small molecule compounds were added to stimulate the reprogramming process.

A non-viral iPSCs induction method including the following steps:

1) Constructing a recombinant plasmid by introducing the DNA sequences expressing the reprogramming factors OCT4, SOX2, GLIS1, KLF4, L-MYC and hsa-miR-302s into an episomal vector;

Wherein the hsa-miR-302s is hsa-miR-302cluster, sequence of hsa-miR-302cluster is shown as SEQ ID No:12;

Wherein, the reprogramming factors OCT4 and GLIS1 are linked through P2A coexpression element and the transcription initiation is through EF-1α promoter, the reprogramming factors KLF4 and SOX2 are linked through P2A coexpression element and the transcription initiation is through EF-1α promoter, the DNA sequences containing genes of OCT4, GLIS1, KLF4 and SOX2 are constructed into an episomal vector together; the transcription initiation of reprogramming factor L-MYC and the transcription initiation of reprogramming factor hsa-miR-302s are through EF-1α promoter and CMV promoter respectively, and then the DNA sequences are constructed into an episomal vector;

2) The recombinant plasmids obtained in step 1) were introduced into human somatic cells, and induced to iPSCs after induction culture for 15 days;

Wherein a mixture of 0.5 μM PD0325901, 3 μM CHIR-99021, 0.25 mM sodium butyrate and 2 μM tranylcypromine hydrochloride was added daily to the induction culture from day 0 to day 8 in the experiment group; and the cell culture fed with the induction medium without adding small molecule compound was used as the control group.

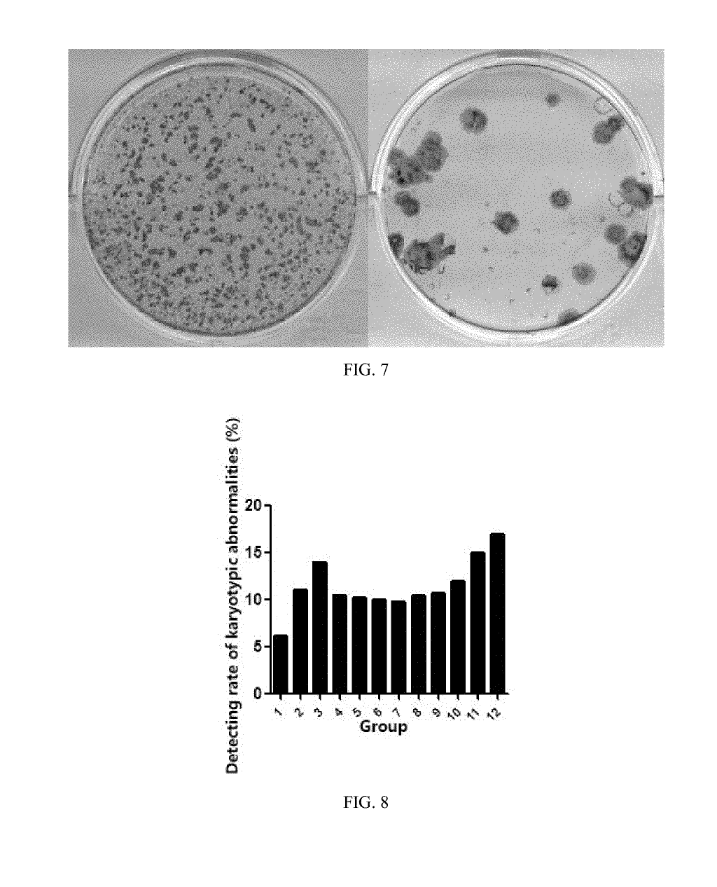

FIG. 7 shows the AP staining of the experimental group and the control group in the Example. It can be seen from FIG. 7 that the induction efficiency of the experiment group is much higher than that of the control group after induction culture, and it is more advantageous to introduce the small molecule compound into an induction culture. The introduction of a small molecule compound can effectively stimulate the induction reprogramming process and improve the induction reprogramming efficiency of iPSCs.

### Example 3

The present example provides a non-viral iPSCs induction method, in which the influences of high risk factors c-MYC, SV40-LT or TP53 on iPSCs were studied in addition to the influences of the reprogramming factors OCT4, SOX2, GLIS1, KLF4, L-MYC and hsa-miR-302s on iPSCs, which could be used to study the influences of different reprogramming factors on the karyotype of iPSCs.

Wherein c-MYC, SV40-LT or TP53 shRNA were construct into an episomal vector respectively, or TP53 siRNA, a TP53 gene inhibitor, was transfected into the somatic cells directly, or Pifithrin-μ, or Pifithrin-α hydrobromide was added into the somatic cells directly.

A non-viral iPSCs induction method including the following steps:

1) The DNA sequences expressing the reprogramming factors OCT4, SOX2, GLIS1, KLF4, L-MYC and hsa-miR-302s were constructed into the episomal vectors which was the control group, i.e. Group 1; On the basis of the control group, experimental groups were constructed into episomal vectors with high risk factors c-MYC, SV40-LT or TP53 shRNA as shown in the following table simultaneously, or constructed by the transfection of TP53 siRNA, a TP53 inhibitor, directly into the somatic cells, or the recombinant plasmid was obtained by adding Pifithrin-μ, or Pifithrin-α hydrobromide into the somatic cells directly, which were Group 2-12 respectively.

TABLE 3
Combination of the high risk reprogramming factors

| | Type | | | | | | | |
|---|---|---|---|---|---|---|---|---|
| | | | | Pifithrin-α | TP53 | TP53 | TP53 | TP53 |
| Group | c-MYC | SV40-LT | Pifithrin-μ | hydrobromide | shRNA1 | shRNA2 | siRNA1 | siRNA2 |
| 1 | − | − | − | − | − | − | − | − |
| 2 | + | − | − | − | − | − | − | − |
| 3 | − | + | − | − | − | − | − | − |
| 4 | − | − | + | − | − | − | − | − |
| 5 | − | − | − | + | − | − | − | − |
| 6 | − | − | − | − | + | − | − | − |
| 7 | − | − | − | − | − | + | − | − |
| 8 | − | − | − | − | − | − | + | − |
| 9 | − | − | − | − | − | − | − | + |
| 10 | + | − | − | − | + | − | − | − |
| 11 | − | + | − | − | + | − | − | − |
| 12 | + | + | − | − | + | − | − | − |

Wherein, the reprogramming factors OCT4 and GLIS1 are linked through P2A coexpression element and the transcription initiation is through EF-1α promoter, the reprogramming factors KLF4 and SOX2 are linked through P2A coexpression element and the transcription initiation is through EF-1α promoter, the DNA sequences containing genes of OCT4, GLIS1, KLF4 and SOX2 are constructed into an episomal vector together; the transcription initiation of reprogramming factor L-MYC and the transcription initiation of reprogramming factor hsa-miR-302s are through EF-1α promoter and CMV promoter respectively, and then the DNA sequences are constructed into an episomal vector. Wherein the hsa-miR-302s is hsa-miR-302cluster, sequence of hsa-miR-302cluster is shown as SEQ ID No:12;

C-MYC, SV40-LT or TP53 shRNA in Table 3 were constructed into another new episomal vector, in which c-MYC and SV40LT were linked by EF1α and thus initiated the transcription, the coexpression elements were P2A; TP53 shRNA was linked by U6 promoter and thus initiated the transcription;

2) The recombinant plasmids obtained in step 1) were introduced into human somatic cells, or cells were transfected with TP53 siRNA, or Pifithrin-μ, or Pifithrin-α hydrobromide were added directly into the somatic cells, and then induced to iPSCs after induction culture for 15 days;

FIG. 8 is a column diagram which shows the detecting rate of karyotypic abnormalities of Group 1-12; As shown in FIG. 8, the rate of karyotypic abnormalities in Group 1 without the addition of any high risk factors or TP53 inhibitory factor was about 6%, which was the lowest; While, the rate of karyotype abnormality in Group 3 with the addition of a high risk reprogramming factor SV40-LT was the highest among the experimental groups with the addition of only one high risk factor; From the comparison of Group 10-11 with Group 12, the rates of karyotypic abnormalities of the experimental groups with the addition of three high risk factors were higher than that of the experimental groups with the addition of two high risk factors.

### Example 4

The present example provides a non-viral iPSCs induction method, which is based on Example 1. For the influences of different small molecule compounds on iPSCs, the induction efficiency was detected by AP staining.

A non-viral iPSCs induction method including the following steps:

1) Constructing a recombinant plasmid by introducing the DNA sequences expressing the reprogramming factors OCT4, SOX2, GLIS1, KLF4, L-MYC and hsa-miR-302s into an episomal vector;

Wherein, the reprogramming factors OCT4 and GLIS1 are linked through P2A coexpression element and the transcription initiation is through EF-1α promoter, the reprogramming factors KLF4 and SOX2 are linked through P2A coexpression element and the transcription initiation is through EF-1α promoter, the DNA sequences containing genes of OCT4, GLIS1, KLF4 and SOX2 are constructed into an episomal vector together; the transcription initiation of reprogramming factor L-MYC and the transcription initiation of reprogramming factor hsa-miR-302s are through EF-1α promoter and CMV promoter respectively, and then the DNA sequences are constructed into an episomal vector; Wherein the hsa-miR-302s is hsa-miR-302cluster, sequence of hsa-miR-302cluster is shown as SEQ ID No:12;

2) The recombinant plasmid obtained in step 1) was introduced into human somatic cells, and induced to iPSCs by adding small-molecule compounds shown in the following table on day 0 to day 8 of the induction culture.

TABLE 4
Combination of small molecule compounds added

| Type | Name | 1 | 2 | 3 | 4 | 5 | 6 | 7 | 8 | 9 | 10 | 11 | 12 | 13 |
|---|---|---|---|---|---|---|---|---|---|---|---|---|---|---|
| MEK inhibitor^{a)} | PD0325901 | + | − | − | − | − | − | − | − | + | − | + | − | + |
| | PD98059 | − | + | − | − | − | − | − | − | − | − | − | − | − |
| GSK-3β | Tideglusib | − | − | + | − | − | − | − | − | − | − | − | − | − |
| inhibitor^{b)} | CHIR-99021 | − | − | − | + | − | − | − | − | + | − | + | + | + |
| | TWS119 | − | − | − | − | + | − | − | − | − | − | − | − | − |
| Histone | Sodium butyrate | − | − | − | − | − | + | − | − | − | + | + | + | + |
| deacetylase | Sodium valproate | − | − | − | − | − | − | + | − | − | − | − | − | − |
| inhibitors^{c)} | | | | | | | | | | | | | | |
| Lysine specific | Tranylcypromine | − | − | − | − | − | − | − | + | − | + | − | + | + |
| demethylasel | hydrochloride | | | | | | | | | | | | | |
| inhibitor^{d)} | | | | | | | | | | | | | | |

Note:
^{a)}The concentrations of MEK inhibitor is 0.5 μM;
^{b)}The concentrations of GSK-3β inhibitor is 3 μM;
^{c)}The concentrations of histone deacetylase inhibitor is 0.25 mM;
^{d)}The concentrations of lysine specific demethylasel inhibitor is 2 μM;

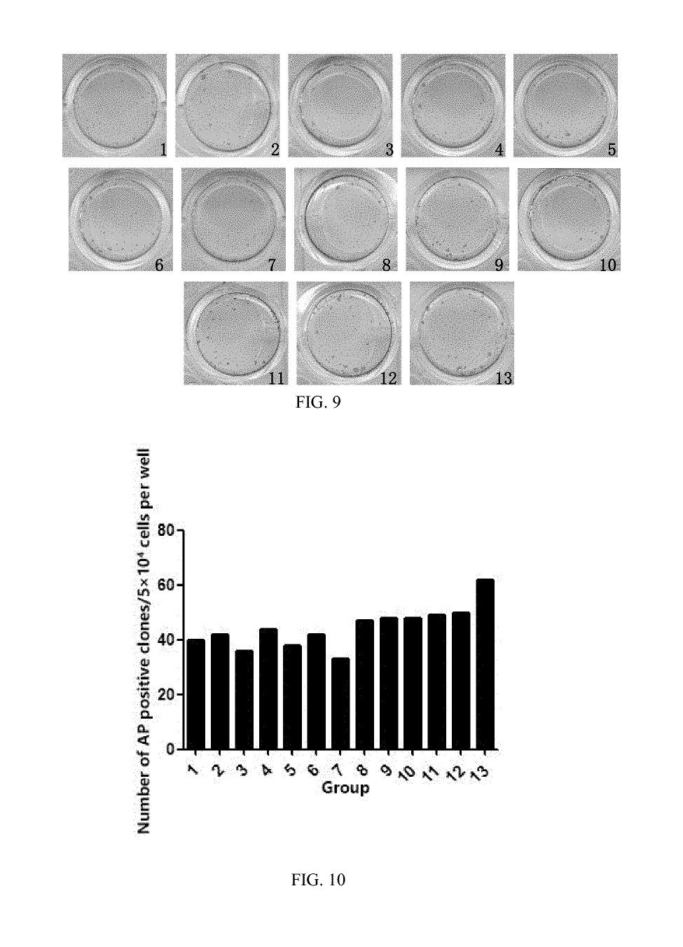

FIG. 9 is a scanning image of AP staining of Group 1-13. FIG. 10 is a column diagram which shows the counting of the AP staining. According to the above mentioned determination of AP staining, it was shown that all 4 types of small molecule compounds disclosed in the present invention promoted cell reprogramming better, wherein the efficiency of positive clones in Group 13 was the highest, i.e., the addition of all 4 types of small molecule compounds simultaneously promote the cell reprogramming better.

### Example 5

The present example provides a non-viral iPSCs induction method, which is based on Example 1. The influences of small molecule compounds with different concentrations on the iPSCs were compared, and then the induction efficiency was detected by AP staining.

A non-viral iPSCs induction method including the following steps:

1) Constructing a recombinant plasmid by introducing the DNA sequences expressing the reprogramming factors OCT4, SOX2, GLIS1, KLF4, L-MYC and hsa-miR-302s into an episomal vector;

Wherein, the reprogramming factors OCT4 and GLIS1 are linked through P2A coexpression element and the transcription initiation is through EF-1α promoter, the reprogramming factors KLF4 and SOX2 are linked through P2A coexpression element and the transcription initiation is through EF-1α promoter, the DNA sequences containing genes of OCT4, GLIS1, KLF4 and SOX2 are constructed into an episomal vector together; the transcription initiation of reprogramming factor L-MYC and the transcription initiation of reprogramming factor hsa-miR-302s are through EF-1α promoter and CMV promoter respectively, and then the DNA sequences are constructed into an episomal vector; Wherein the hsa-miR-302s is hsa-miR-302cluster, sequence of hsa-miR-302cluster is shown as SEQ ID No:12;

2) The recombinant plasmid obtained in step 1) was introduced into human somatic cells, and induced to iPSCs by adding small-molecule compounds at the concentrations shown in the following table on day 0 to day 8 of the induction culture.

TABLE 5
Influences on the induction culture of the small molecule compounds at
different concentrations

| | Concentration | | | |
|---|---|---|---|---|
| | | | Sodium | Tranylcypromine |
| | PD0325901 | CHIR-99021 | butyrate | hydrochloride |
| Group | (μM) | (μM) | (mM) | (μM) |
| 1 | 0.1 | — | — | — |
| 2 | 0.25 | — | — | — |
| 3 | 0.5 | — | — | — |
| 4 | 2 | — | — | — |
| 5 | — | 0.1 | — | — |
| 6 | — | 1 | — | — |
| 7 | — | 3 | — | — |
| 8 | — | 6 | — | — |
| 9 | — | — | 0.05 | — |
| 10 | — | — | 0.1 | — |
| 11 | — | — | 0.25 | — |
| 12 | — | — | 2 | — |
| 13 | — | — | — | 0.1 |
| 14 | — | — | — | 1 |
| 15 | — | — | — | 2 |
| 16 | — | — | — | 10 |

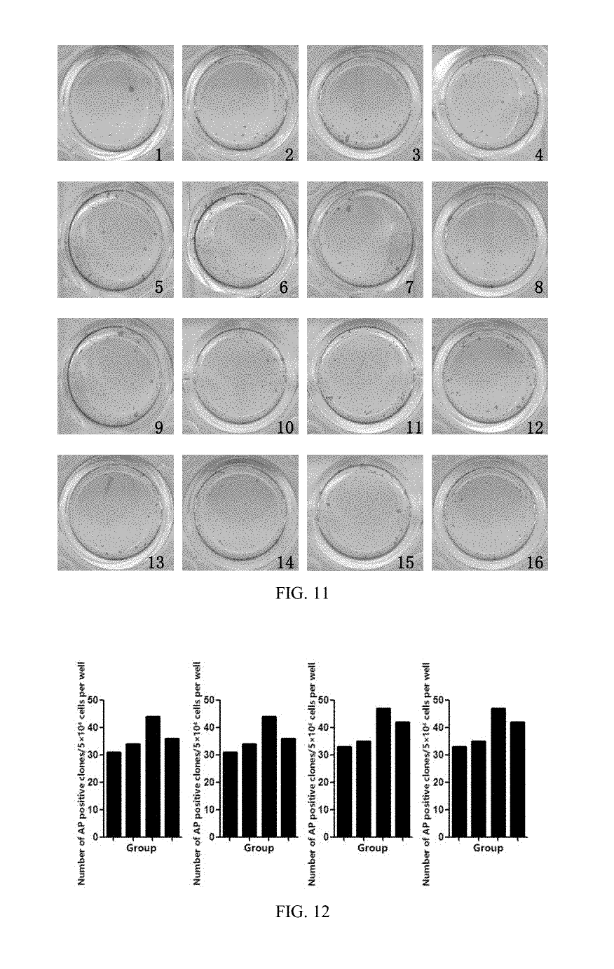

The scanning image of AP staining of Groups 1-16 are shown in FIG. 11, and the histogram of the AP staining is shown in FIG. 12. According to the above-mentioned AP staining results, it is indicated that all the small molecule compounds with different concentration added could promote the cell reprogramming process during the induction culture. PD0325901 is preferably at a concentration of 0.5 μM, CHIR-99021 is preferably at a concentration of 3 μM, the sodium butyrate is preferably at a concentration of 0.25 μM, and the tranylcypromine hydrochloride is preferably at a concentration of 2 μM.

### Example 6

The present example provides a non-viral iPSCs induction method, which is based on Example 1. The influences of the addition time of small molecule compounds on the culture of iPSCs were compared, and then the induction efficiency was detected by AP staining.

A non-viral iPSCs induction method including the following steps:

1) Constructing a recombinant plasmid by introducing the DNA sequences expressing the reprogramming factors OCT4, SOX2, GLIS1, KLF4, L-MYC and hsa-miR-302s into an episomal vector;

Wherein, the reprogramming factors OCT4 and GLIS1 are linked through P2A coexpression element and the transcription initiation is through EF-1α promoter, the reprogramming factors KLF4 and SOX2 are linked through P2A coexpression element and the transcription initiation is through EF-1α promoter, the DNA sequences containing genes of OCT4, GLIS1, KLF4 and SOX2 are constructed into an episomal vector together; the transcription initiation of reprogramming factor L-MYC and the transcription initiation of reprogramming factor hsa-miR-302s are through EF-1α promoter and CMV promoter respectively, and then the DNA sequences are constructed into an episomal vector; Wherein the hsa-miR-302s is hsa-miR-302cluster, sequence of hsa-miR-302cluster is shown as SEQ ID No:12;

2) The recombinant plasmids obtained in step 1) were introduced into human somatic cells, and the cells were cultured with an induction. A mixture of 0.5 μM PD0325901, 3 μM CHIR-99021, 0.25 μM sodium butyrate and 2 μM tranylcypromine hydrochloride was added daily at the times indicated in the following table, and thus obtained the iPSCs.

| | Tim | | | | | | | | | | | | |
|---|---|---|---|---|---|---|---|---|---|---|---|---|---|
| Group | D0 | D1 | D2 | D3 | D4 | D5 | D6 | D7 | D8 | D9 | D10 | D11 | D12 |
| 1 | + | + | + | − | − | − | − | − | − | − | − | − | − |
| 2 | + | + | + | + | + | − | − | − | − | − | − | − | − |
| 3 | + | + | + | + | + | + | + | − | − | − | − | − | − |
| 4 | + | + | + | + | + | + | + | + | + | − | − | − | − |
| 5 | + | + | + | + | + | + | + | + | + | + | + | − | − |
| 6 | + | + | + | + | + | + | + | + | + | + | + | + | + |
| 7 | − | − | + | + | + | + | + | + | + | + | + | + | + |
| 8 | − | − | − | − | + | + | + | + | + | + | + | + | + |
| 9 | − | − | − | − | − | − | + | + | + | + | + | + | + |
| 10 | − | − | − | − | − | − | − | − | + | + | + | + | + |
| 11 | − | − | − | − | − | − | − | − | − | − | + | + | + |

Note:
D for days, D0 for test day, and so on.

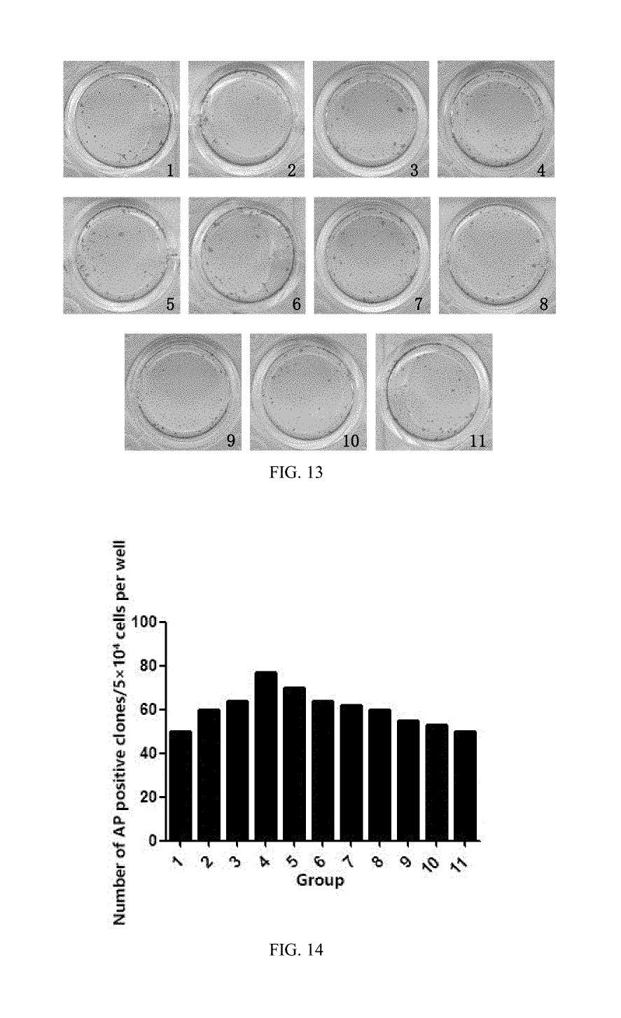

The scanning picture of AP staining of Groups 1 to 11 are shown in FIG. 13, and the histogram of the AP staining is shown in FIG. 14. According to the above-mentioned AP staining results, it is indicated that there was no significant influence of the time of addition of a small molecule compounds to the cell reprogramming process during the induction culture. The positive clones of Group 4 were the most efficient, that is, the ideal time of adding the small molecule compound was from the starting day till the 8th day of an induction culture.

### Example 7

The present example provides a non-viral iPSCs induction method, wherein the influences of adding hsa-miR-302s with different lengths on the culture of iPSCs were compared, and then the induction efficiency was detected by AP staining.

A non-viral iPSCs induction method including the following steps:

1) Constructing a recombinant plasmid by introducing the DNA sequences expressing the reprogramming factors OCT4, SOX2, GLIS1, KLF4, L-MYC and hsa-miR-302s into an episomal vector;

Wherein, the reprogramming factors OCT4 and GLIS1 are linked through P2A coexpression element and the transcription initiation is through EF-1α promoter, the reprogramming factors KLF4 and SOX2 are linked through P2A coexpression element and the transcription initiation is through EF-1α promoter, the DNA sequences containing genes of OCT4, GLIS1, KLF4 and SOX2 are constructed into an episomal vector together; the transcription initiation of reprogramming factor L-MYC and the transcription initiation of reprogramming factor hsa-miR-302s are through EF-1α promoter and CMV promoter respectively, and then the DNA sequences are constructed into an episomal vector;

Wherein, the information of hsa-miR-302s is as follows:

TABLE 6
hsa-miR-302s information

| Group | hsa-miR-302s |
|---|---|
| 1 | hsa-miR-302b(5′ + 75 bp, 3′ + 27 bp) |
| 2 | hsa-miR-302b(5′ + 150 bp, 3′ + 54 bp) |
| 3 | hsa-miR-302c(5′ + 27 bp, 3′ + 56 bp) |
| 4 | hsa-miR-302c(5′ + 54 bp, 3′ + 111 bp) |
| 5 | hsa-miR-302a(5′ + 55 bp, 3′ + 56 bp) |
| 6 | hsa-miR-302a(5′ + 111 bp, 3′ + 111 bp) |
| 7 | hsa-miR-302d(5′ + 55 bp, 3′ + 31 bp) |
| 8 | hsa-miR-302d(5′ + 111 bp, 3′ + 62 bp) |
| 9 | hsa-miR-302bcad(5′ + 75 bp, 3′ + 31 bp) |
| 10 | hsa-miR-302bcad(5′ + 150 bp, 3′ + 62 bp) |
| 11 | hsa-miR-302cluster(5′ + 75 bp, 3′ + 130 bp) |
| 12 | hsa-miR-302cluster(5′ + 150 bp, 3′ + 260 bp) |

2) The recombinant plasmids obtained in step 1) were introduced into human somatic cells, and induced to iPSCs after an induction culture, and a mixture of 0.5 μM PD0325901, 3 μM CHIR-99021, 0.25 μM sodium butyrate and 2 μM tranylcypromine hydrochloride was added daily to the induction culture from day 0 to day 8.

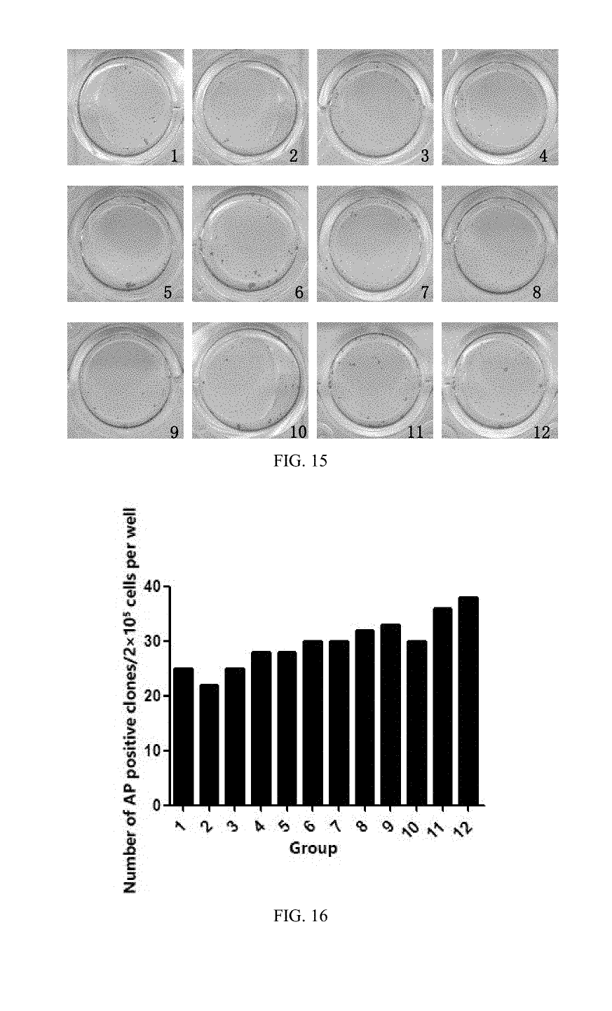

The scanning picture of AP staining of Groups 1 to 12 are shown in FIG. 15, and the histogram of the AP staining is shown in FIG. 16. According to the above-mentioned AP staining results, it is indicated that there was no significant influence of the lengths of hsa-miR-302s to the cell reprogramming process. Wherein, the hsa-miR-302bcad in Group 9-10 were the combinations of hsa-miR-302b, hsa-miR-302c, hsa-miR-302a and hsa-miR-302d, which had a higher efficiency of the positive clones than that of Group 1-8 which were only single hsa-miR-302s; Group 11-12 were based on the above mentioned combinations with an addition of hsa-miR-367, and their efficiency of positive clones obtained by induction culture were much higher.

### Example 8

The present example provides a non-viral iPSCs induction method wherein the influences of promoters on the culture of iPSCs were compared, and then the induction efficiency was detected by AP staining.

1) Constructing a recombinant plasmid by introducing the DNA sequences expressing the reprogramming factors OCT4, SOX2, GLIS1, KLF4, L-MYC and hsa-miR-302s into an episomal vector;

Wherein, the transcription initiation of the expression reprogramming factors OCT4 and GLIS1 were through a linkage to a promoter by a P2A coexpression element, the transcription initiation of the expression reprogramming factors KLF4 and SOX2 were through a linkage to a promoter by a P2A coexpression element, the DNA sequences of OCT4, GLIS1, KLF4 and SOX2 were constructed into an episomal vector together; L-MYC and hsa-miR-302s were constructed into another episomal vector.

Wherein, the encoding genes (OCT4, SOX2, GLIS1, KLF4 and L-MYC) of expressed protein and the non-encoding gene hsa-miR-302s of expressed protein were linked by a promoter as shown in the following table, and thus initiated the transcription; Wherein, the hsa-miR-302s is a hsa-miR-302cluster, sequence of hsa-miR-302cluster is shown as SEQ ID No:12;

TABLE 7
Combination of promoters

Combination of promoters

| | Expression of encoding | hsa-miR-302s |
|---|---|---|
| Group | proteins Gene promoter | promoter |
| 1 | EF-1a | CMV |
| 2 | EF-1a | EF-1a |
| 3 | CMV | CMV |
| 4 | CAG | CMV |
| 5 | EF-1a | U6 |
| 6 | EF-1a | H1 |

2) The recombinant plasmids obtained in step 1) were introduced into human somatic cells, and induced to iPSCs after an induction culture for 15 days, wherein a mixture of 0.5 μM PD0325901, 3 μM CHIR-99021, 0.25 μM sodium butyrate and 2 μM tranylcypromine hydrochloride was added daily to the induction culture from day 0 to day 8.

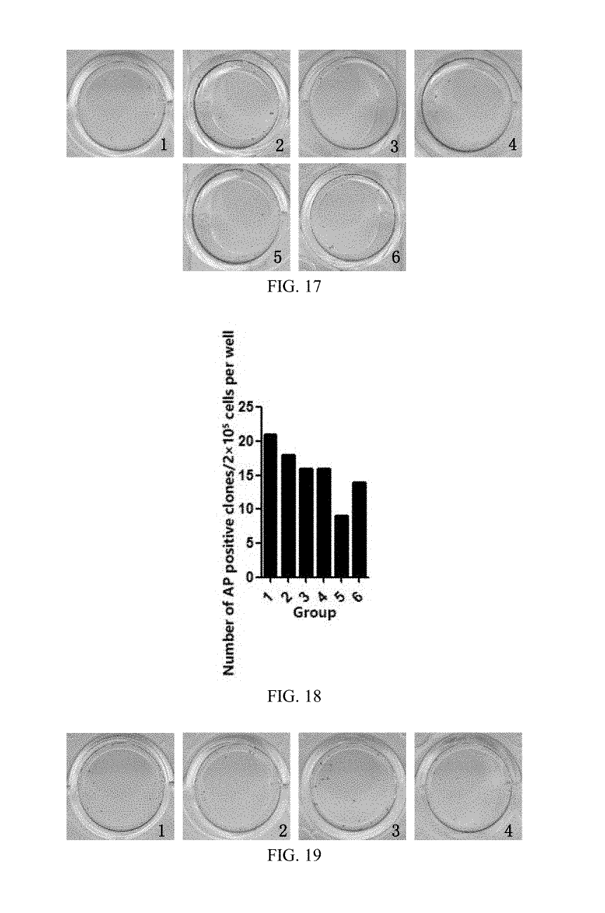

The scanning picture of AP staining of Groups 1 to 6 are shown in FIG. 17, and the histogram of the AP staining is shown in FIG. 18; According to the result in FIG. 18, it is indicated that all the plasmids constructed by different promoters could induce the cell reprogramming; The efficiency of positive clones was highest when the promoter EF-1a was used for the linkage and the transcription initiation of genes encoding the expressed protein, and the promoter CMV was used for the linkage and the transcription initiation of hsa-miR-302s at the same time.

### Example 9

The present example provides a non-viral iPSCs induction method wherein the influences of different coexpression elements on the culture of iPSCs were compared, and then the induction efficiency was detected by AP staining.

1) Constructing a recombinant plasmid by introducing the DNA sequences expressing the reprogramming factors OCT4, SOX2, GLIS1, KLF4, L-MYC and hsa-miR-302s into an episomal vector;

Wherein, the coexpression elements are shown in the following table when the reprogramming factors OCT4, SOX2, GLIS1 and KLF4 were in a coexpression of reprogramming factor genes in expressing two or more expression proteins through a single promoter.

TABLE 8
combination of coexpression elements

| | Type |
|---|---|
| Group | Coexpression element |
| 1 | IRES1 |
| 2 | IRES2 |
| 3 | P2A |
| 4 | F2A |

Wherein, the DNA sequences of OCT4, GLIS1, KLF4 and SOX2 were constructed into an episomal vector together, and L-MYC and hsa-miR-302s were constructed into another episomal vector at the same time, during their process of construction to the episomal vectors. Wherein, the encoding genes (OCT4, SOX2, GLIS1, KLF4 and L-MYC) of expressed protein were linked by a promoter EF-1a, and thus initiated the transcription, and the non-encoding gene hsa-miR-302s of expressed protein were linked by a promoter CMV, and thus initiated the transcription; Wherein, the hsa-miR-302s was a hsa-miR-302cluster, sequence of hsa-miR-302cluster is shown as SEQ ID No:12;

2) The recombinant plasmids obtained in step 1) were introduced into human somatic cells, and induced to iPSCs after an induction culture for 15 days, wherein a mixture of 0.5 PD0325901, 3 μM CHIR-99021, 0.25 μM sodium butyrate and 2 μM tranylcypromine hydrochloride was added daily to the induction culture from day 0 to day 8.

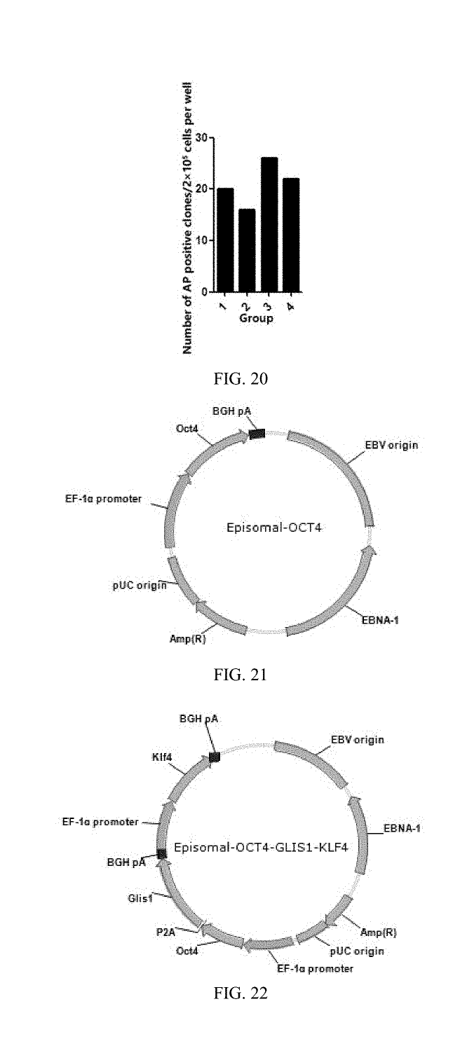

The scanning image of AP staining of Groups 1-4 are shown in FIG. 19, and the histogram of the AP staining is shown in FIG. 20; According to the result in FIG. 20, it is indicated that all the plasmids constructed by different coexpression elements could induce cell reprogramming; The efficiency of positive clones was highest in Group 3, i.e. the efficiency of cell reprogramming was higher when P2A coexpressing elements were used.

### Example 10

The present example provides a non-viral iPSCs induction method wherein the influences of the combinations of reprogramming factors and episomal vectors on the culture of iPSCs were compared, and then the induction efficiency was detected by AP staining.

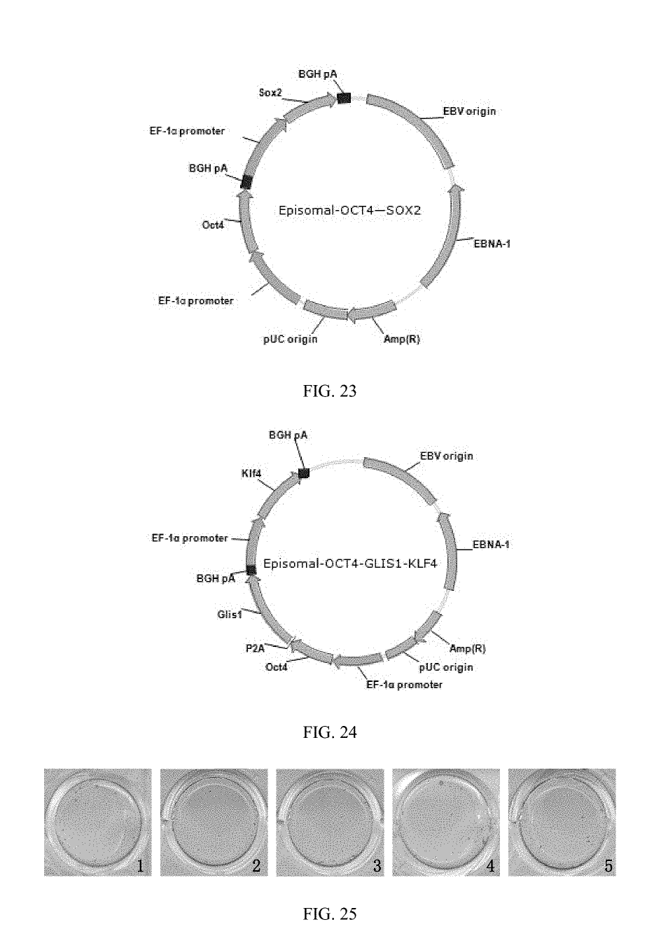

1) Constructing a recombinant plasmid by introducing the DNA sequences expressing the reprogramming factors OCT4, SOX2, GLIS1, KLF4, L-MYC and hsa-miR-302s into an episomal vector;

Wherein, the reprogramming factors (OCT4, SOX2, GLIS1, KLF4 and L-MYC) in a coexpression of reprogramming factor genes in expressing two or more expression proteins were linked to a promoter through a P2A coexpression elements and thus initiated the transcription; The example maps of the recombinant plasmids in Groups 1 and 3-5 are shown in FIGS. 21-24;

TABLE 9
Combination of reprogramming factors and episomal vectors

| | Number of | | | | | | |
|---|---|---|---|---|---|---|---|
| | recombinant | | | | | | |
| Group | plasmids | Combination of reprogramming factors | | | | | |
| 1 | 6 | OCT4 | GLIS1 | KLF4 | SOX2 | L-MYC | hsa-miR-302s |
| 2 | 6 | OCT4 | GLIS1 | KLF4^{a)} | SOX2 | L-MYC | hsa-miR-302s |

| | | | |
|---|---|---|---|
| 3 | 2 | OCT4, GLIS1, KLF4 | SOX2, L-MYC, hsa-miR-302s |
| 4 | 2 | OCT4, GLIS1, KLF4, | L-MYC, hsa-miR-302s |
| | | SOX2 | |

| | | |
|---|---|---|
| 5 | 1 | OCT4, GLIS1, KLF4, SOX2, L-MYC, hsa-miR-302s |

Note:
^{a)}The length of KLF4 here is 1440 bp, and the length of KLF4 is 1413 bp unless otherwise specified;

Wherein, the encoding genes (OCT4, SOX2, GLIS1, KLF4 and L-MYC) of expressed protein were linked by a promoter EF-1a, and thus initiated the transcription, and the non-encoding gene hsa-miR-302s of expressed protein were linked by a promoter CMV, and thus initiated the transcription, during their process of construction to the episomal vectors; Wherein, the hsa-miR-302s was a hsa-miR-302cluster, sequence of hsa-miR-302cluster is shown as SEQ ID No:12;

2) The recombinant plasmids obtained in step 1) were introduced into human somatic cells, and induced to iPSCs after an induction culture for 15 days, wherein a mixture of 0.5 PD0325901, 3 μM CHIR-99021, 0.25 μM sodium butyrate and 2 μM tranylcypromine hydrochloride was added daily to the induction culture from day 0 to day 8.

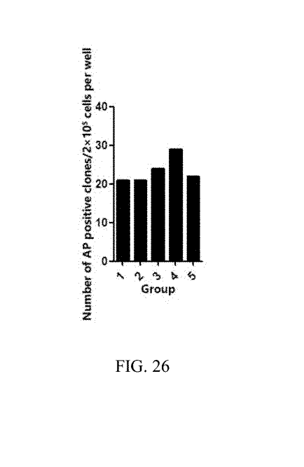

The scanning picture of AP staining of Groups 1 to 5 are shown in FIG. 25, and FIG. 26 shows their efficiency of positive clones; As shown in FIG. 26, the cell reprogramming could be performed by the constructing of the reprogramming factors into a different number of episomal vectors; Wherein, the efficiency of positive clones was highest in Group 4, i.e. the efficiency of cell reprogramming was highest when the DNA sequences of OCT4, GLIS1, KLF4 and SOX2 were constructed into an episomal vector together, and L-MYC and hsa-miR-302s were constructed into another episomal vector at the same time.

It will be apparent to those skilled in the art that various variants and modifications can be made according to the technical scheme described above without departing from the spirit and scope of the invention as defined by the appended claims.

We claim:

1. Induced pluripotent stem cells, wherein the Induced pluripotent stem cells are obtained by the following steps:
a) constructing recombinant plasmids by the introduction of DNA sequences encoding reprogramming factors POU5F1, SOX2, GLIS1, KLF4, MYCL and hsa-miR-302s into episomal vectors, wherein link and transcription initiation of the reprogramming factors are through promoters;
wherein, the hsa-miR-302s DNA sequence comprises one or more sequences selected from hsa-miR-302a, hsa-miR-302b, hsa-miR-302c and hsa-miR-302d; and
b) introducing the recombinant plasmids constructed in step a) into human somatic cells to reprogram into iPSCs in an induction culture.

2. The induced pluripotent stem cells according to claim 1, wherein the hsa-miR-302s DNA sequence in step a) further comprises a DNA sequence of hsa-miR-367, wherein the DNA sequence of hsa-miR-367 comprises mature sequence of has-miR-367.

3. The induced pluripotent stem cells according to claim 1, wherein the link and transcription initiation of POU5F1, SOX2, GLIS1, KLF4 and MYCL in step a) are through a RNA polmerase II promoter (pol II promoter).

4. The induced pluripotent stem cells according to claim 1, wherein the link and transcription initiation of POU5F1, SOX2, GLIS1, KLF4 and MYCL in step a) are through a protomer of the protomers, and the protomer is selected from EF-1α promoter, CMV promoter and CAG promoter.

5. The induced pluripotent stem cells according to claim 1, wherein the link and transcription initiation of hsa-miR-302s in step a) are through a promoter of the protomers, and the promoter is selected from RNA polmerase I promoter, RNA polmerase II promoter, and RNA polmerase III promoter.

6. The induced pluripotent stem cells according to claim 1, wherein the link and transcription initiation of hsa-miR-302s in step a) are through a promoter of the promoters, the promoter is selected from CMV promoter, U6 promoter and H1 promoter.

7. The induced pluripotent stem cells according to claim 1, wherein IRES elements and 2A elements are used to coexpress the reprogramming factors POU5F1, SOX2, GLIS1, KLF4 and MYCL in step a) DNA sequences of the reprogramming factors expressing two or more proteins are coexpressed through a single promoter of the promoters.

8. The induced pluripotent stem cells according to claim 7, wherein IRES elements comprises IRES1 element and IRES2 element, and 2A elements comprises P2A element and F2A element.

9. The induced pluripotent stem cells according to claim 1, wherein, the reprogramming factors POU5F1 and GLIS1 in step a) are linked through P2A element and the transcription initiation is through EF-1α promoter, the reprogramming factors KLF4 and SOX2 are linked through P2A element and the transcription initiation is through EF-1α promoter, the DNA sequences containing genes of SOX2, GLIS1, KLF4 and POU5F1 are constructed into a first episomal vector of the episomal vector together; the transcription initiation of reprogramming factor MYCL and the transcription initiation of reprogramming factor hsa-miR-302s are through EF-1α promoter and CMV promoter respectively, and then the DNA sequences are constructed into a second episomal vector of the episomal vector.

10. The induced pluripotent stem cells according to claim 1, wherein a small molecule compound is added during the induction culture in step b), the small molecule compound is one or more molecules selected from MEK inhibitors, GSK-3β inhibitors, histone deacetylase inhibitors and lysine specific demethylasel inhibitors.

11. The induced pluripotent stem cells according to claim 10, wherein the small molecule compound in step b) is a combination of PD0325901, CHIR-99021, sodium butyrate and tranylcypromine hydrochloride.

12. The induced pluripotent stem cells according to claim 11, where in step b), a concentration of PD0325901 is in a range of 0.1-2 μM, a concentration of CHIR-99021 is in a range of 0.1-6 μM, a concentration of sodium butyrate is in a range of 0.05-2 mM, and a concentration of tranylcypromine hydrochloride is in a range of 0.1-10 μM.

13. The induced pluripotent stem cells according to claim 10, where in step b), the small molecule compound is added on any one or more days from day 0 to day 12 during the induction culture.

14. The induced pluripotent stem cells according to claim 10, where in step b), the small molecule compound is added every day from day 0 to day 8 during the induction culture.
